(19) United States
(12) Patent Application Publication
(10) Pub. No.: US 2006/0127499 A1
(43) Pub. Date: Jun. 15, 2006

(54) Vitamin-mineral compositions

(76) Inventors: Mikhail I. Lazarev, Moscow (RU); Rustam K. Yenileyev, Moscow (RU)

Correspondence Address: LANIER FORD SHAVER & PAYNE, P O BOX 2087, HUNTSVILLE, AL, 35804, US

(21) Appl. No.: 11/346,895

(22) Filed: Feb. 3, 2006

Related U.S. Application Data
(63) Continuation of application No. 10/343,781, filed on Oct. 17, 2003.

(30) Foreign Application Priority Data
Feb. 14, 2001 (RU) 2001104090

(51) Int. Cl. A61K 33/36; A61K 33/32; A61K 33/26; A61K 33/04; A61K 33/34

(52) U.S. Cl. 424/638; 424/641; 424/646; 424/667; 424/682; 424/702; 514/168; 514/52; 514/251; 514/276; 514/350; 514/356; 514/474; 514/458; 514/383; 514/725; 514/682

(57) ABSTRACT

This invention provides compositions of vitamins and minerals used in the prevention and treatment of vitamin and mineral deficiency. These compositions provide a minimization of the intake dosage of vitamins and minerals while still meeting the nutritional daily requirements for these vitamins and minerals. At the same time, these compositions are easier to manufacture, have an improved prolonged shelf-life, and are easy and convenient to use. These compositions comprise 3 to 5 dose formulations that are ingested separately. Each dose formulation contains vitamins and minerals that are compatible with each other regarding systemic absorption and storage shelf-life.

### REFERENCE TO RELATED APPLICATIONS

The present application is a continuation of and claims priority to non-provisional patent application Ser. No. 10/343,781, entitled “Vitamin-Mineral Compositions,” which is fully incorporated herein by reference, and which was a national stage application of and claims priority to International Application No. PCT/RU2002/000034, filed Feb. 5, 2002, which claims the benefit of Russian Application No. 2001104090, filed Feb. 14, 2001. The present application also claims such priority.

### BACKGROUND OF INVENTION

1. Field of the Invention

This invention relates to vitamin and mineral compositions, and, more particularly, to vitamin-mineral compositions that are synergistic while avoiding negative interactions.

2. Technical Background

Vitamin-mineral compositions usually have several vitamins and minerals in a single dose formulation. For example, Complivit contains 19 components comprising 12 vitamins or vitamin-like substances (A, E, C, B₁, B₅, B₆, B₉, B₁₂, riboflavin, nicotinamide, rutin, and lipoic acid) and 7 minerals (iron, copper, calcium, cobalt, manganese, zinc, and magnesium). The disadvantage of Complivit is the lack of compatibility between some of its components, either during long-term storage or during absorption into the body.

In an effort to avoid incompatibility, vitamins and minerals have been divided into two separate formulations, for example, Duovit comprises two separate differently colored formulations. When taken separately over a period of 24 hours, the daily need of the body for vitamins and minerals is satisfied. The first colored formulation contains 10 vitamins (A, B₁, B₂, B₅, B₆, B₉, B₁₂, C, D, and E) and the second colored formulation contains 8 minerals (magnesium, calcium, phosphorus, iron, zinc, copper, manganese, and molybdenum).

In vitamin-mineral compositions such as these it is known that some components interfere with the activity of other components. It is known that zinc reduces uptake of copper and compete with iron and calcium in absorption. Furthermore, iron and calcium reduce the uptake of manganese. Thus, combining all these minerals into one dose formulation is not a rational approach to the delivery of minerals. In order to still meet the daily requirement of the body in this type of formulation, the amount of the minerals in the dosage formulation needs to be increased. This disadvantage of Duovit is typical of most vitamin-mineral dose formulations. Dividing the vitamins and minerals between just two separate dose formulations is inadequate to effectively eliminate negative interactions among the vitamins and minerals.

### SUMMARY OF THE INVENTION

The present invention is a composition of vitamins and minerals to be consumed in separate dose formulations based upon the influence of vitamins and minerals on each other. This composition provides a minimization of the daily dosage of vitamins and minerals while the daily requirements of the body for vitamins and minerals are met. The composition provides a convenient means to consume vitamins and minerals while avoiding the negative interactions of the vitamins and minerals on each other with regard to their uptake into the body. The simplicity of the dose formulations makes production easier, and the unique combination in each dose formulation improves stability and shelf-life. The effect of the composition of the present invention is the increased assimilation into the body of the components contained in the separate dose formulations of the composition. The increased assimilation is due to combining the vitamins and minerals to promote the synergistic interactions of the vitamins and minerals and preventing their antagonistic interactions. This is achieved by the composition comprising three to five different, separate dose formulations. The vitamins and minerals are also divided among the dose formulations to take into consideration the age and food consumption preferences of the user.

### DETAILED DESCRIPTION OF THE PREFERRED EMBODIMENTS

While the following description details the preferred embodiments of the present invention, it is to be understood that the invention is not limited in its application to the details of construction and arrangement of the parts illustrated in the accompanying drawings, since the invention is capable of other embodiments and of being practiced in various ways.

Vitamins and minerals can interact with each other while being produced, stored, absorbed, or acting within the body. Thus, they need to be divided into three to five dose formulations having different vitamin-mineral contents. These different, separate dose formulations are taken separately at different times to exclude or reduce these interactions and to avoid reduced effectiveness. The different dose formulations can be color-coded and placed in blister packs so that a user knows when to consume a given dose formulation during the day.

In a preferred embodiment, a first dose formulation would contain, at a minimum, a combination of vitamin E and selenium; a second would contain, at a minimum, a combination of vitamin K and vitamin D, or vitamin K and calcium; and a third would contain, at a minimum, a combination of iron and copper. The fourth dose formulation may contain zinc and manganese. Various compositions may be made up of the various dose formulations, but each of the aforesaid combination of vitamins and minerals must be contained in only one of the dose formulations of the composition. The composition is then made into 3 to 5 coded formulations to be taken separately to exclude or reduce negative interactions.

It is known that combination preparations or compositions, such as estrogen-progesterones, have color coded dose formulations containing various components (M.D. Mashkousky, Lekarstvennye Sredstva [Medicinal Drugs] ₁₄th Ed., V.2, Moscow, Novaya Volna [New Wave] Publishers, 2000, pp. 49-55). However, the number of dose formulations in such compositions and their contents are determined, not by the necessity to exclude unwanted chemical and physiological interactions, while bringing together synergistic interactions, but by the necessity for a stage-by-stage effect on the human body as determined by the body's condition. Thus, while certain characteristics of these known preparations are similar to those described herein, their technical effects differ from the present invention because they are not designed to enhance synergistic interactions and eliminate negative interactions. The applicants of the present invention discovered the necessity of organizing the various vitamins and minerals into a composition of separate dose formulations, with the composition comprising at least 3 dose formulations capable of meeting the requirements of the human body for vitamins and minerals. For example, iron, manganese, and calcium are essential for the body. However, iron and calcium impede the absorption of manganese. This means that manganese must be placed in a dose formulation that does not contain iron and calcium. Likewise, calcium impedes the absorption of iron (see V. Griffith, Vitamins, Herbs. Minerals, and Food Additives. Moscow, Grand Publishers, 2000), and calcium and iron must also be placed in separate dose formulations. Based upon these negative interactions for iron, calcium, and manganese, a composition containing these minerals must have at least 3 different dose formulations, one containing iron but not calcium and manganese, one containing calcium but not iron or manganese, and one containing manganese but not calcium or iron.

Excessive iron may result in a deficiency of copper (see A. V. Skalny, Human Microelementosis, Moscow Nauchny Min [The World of Science] Publishers, 1999), and a composition of 4 or 5 formulations, instead of 3, can avoid this interaction. However, dose formulations in excess of 5 are not practical for daily use, and doses of 3 to 5 appear to be optimum. The applicants, through their analysis and experimentation, thus, discovered the characteristics of the total number of vitamins and minerals and combinations thereof to produce the various dose formulations of the compositions of the present invention. The composition of the present invention is distinguished by the specific ingredients with their specific uses in a given dose formulation, the manner in which the specific dose formulations are produced, the shape the dose formulations assume, the sequential order in which they are consumed, and the combined features of the dose formulations that form the compositions.

In a preferred embodiment of the present invention, 13 vitamins and 10 minerals are divided into 3 dose formulations to be taken at different times over a 24 hour period. A first dose formulation contains vitamins A, C, and E and magnesium, manganese, selenium, and zinc. A second dose formulation contains vitamins B₂, B₅, B₆, B₉, B₁₂, D₃, K, and biotin (H) and chromium and calcium. A third dose formulation contains vitamins B₁, B₃, and B₆ plus iron, copper, molybdenum, and iodine. These 3 dose formulations are depicted in Table 1. The dose formulations can be produced in any suitable form (tablets, capsules, liquid, etc.) by suitable methods well-known in the art. The dose formulations may also contain any desired excipients and additives, such as, for example, calcium stearate, water, gelatin, nipagin, stearic acid, lactose, starch, talcum, and food colors (E128, 2 G red, E-129.2, AC red charmine, E-131, V patented blue, E-133, and FCF glossyblue). Each dose formulation is specifically coded, such as, for

TABLE 1

| Dose Formulation 1 | | | Dose Formulation 2 | | | Dose Formulation 3 | | |
|---|---|---|---|---|---|---|---|---|
| vitamins: | | | vitamins: | | | vitamins: | | |
| A | 3333 | IU | B2 | 1.7 | mg | B1 | 1.5 | mg |
| E | 10 | IU | B5 | 5 | mg | B3 | 20 | mg |
| C | 80 | mg | B6 | 1 | mg | B6 | 1 | mg |
| | | | B9 | 200 | μg | | | |
| | | | B12 | 3 | μg | | | |
| | | | D3 | 100 | IU | | | |
| | | | H | 30 | μg | | | |
| | | | K1 | 25 | μg | | | |
| minerals: | | | Minerals: | | | minerals: | | |
| Magnesium | 40 | mg | Calcium | 100 | mg | Iron | 18 | mg |
| Manganese | 2.5 | mg | Chromium | 25 | μg | Iodine | 150 | μg |
| Selenium | 25 | μg | | | | Copper | 2 | mg |
| Zinc | 15 | mg | | | | Molybdenum | 250 | μg |

example, by a different color and is placed in a blister pack so that each blister pack contains dose formulations of the same color.

As noted above, a composition may also contain 4 or 5 dose formulations. In the case of 4 dose formulations, the first may contain vitamins B₁, B₃, B₆, and iron and molybdenum; the second copper and iodine; the third vitamins B₂, B₅, B₆, B₉, B₁₂, D₃, K, and biotin and calcium and chromium; and the fourth vitamins A, C, and E, and magnesium, manganese, selenium, and zinc. In the case of 5 dose formulations, the first may contain vitamins B₁, B₃ B₆, and iron and molybdenum; the second copper and iodine; the third vitamins B₂, B₉, B₁₂, D₃, and K, and calcium, phosphorus, manganese, and zinc; the fourth vitamins B₅, B₆, H, and chromium; and the fifth vitamins A, C, and E and manganese and selenium.

The composition of the present invention minimizes the dosage of vitamins and minerals necessary to meet the body's requirements for vitamins and minerals. This is because the negative effects on absorption have been significantly, if not completely, eliminated by dividing the vitamins and minerals among the separate dose formulations. The composition further retains the synergistic effects of vitamin E and selenium plus vitamin K and calcium. Thus, the composition of the present invention simultaneously reduces negative interactions and provides synergistic interactions of vitamins and minerals. Because of improved absorption and bioavailability of vitamins and minerals in the composition of the present invention, daily dosages are reduced 1.5 to 2.5 times compared to standard vitamin-mineral preparations. Reduced daily intake of vitamins and minerals, while meeting daily requirements, is beneficial to users, especially those that may have metabolic disorders. The composition of the present invention is also convenient to use because the dosage formulations can be coded and packaged so that it is easy for a user to take a given dose formulation at a given time.

The composition of the present invention is easier to manufacture and produce compared to standard formulations because there is no need to include certain additives to compensate for and protect against the negative interactions among the vitamins and minerals. The labor intensity of production is expected to decrease by 5%-10% or more. Likewise, the shelf-life of the compositions of the present invention are expected to increase by a factor of 2 or 3, since there are no incompatible components in any of the dose formulations.

The foregoing description has been limited to specific embodiments of this invention. It will be apparent, however, that variations and modifications may be made by those skilled in the art to the disclosed embodiments of the invention, with the attainment of some or all of its advantages and without departing from the spirit and scope of the present invention. It will be understood that various changes in the details, materials, and arrangements of the parts which have been described and illustrated above in order to explain the nature of this invention may be made by those skilled in the art without departing from the principle and scope of the invention as recited in the following claims.

What is claimed is:

1. A multiple dose vitamin-mineral complex to be administered daily, consisting essentially of:
(a) vitamins A, B₁, B₂, B₆, B₁₂, C, D, E, K, biotin, nicotinamide, pantothenic acid, folic acid; and
(b) minerals Fe, I, Cu, Mo, Mg, Mn, Se, Zn, Ca, Cr;
wherein said vitamin-mineral complex is subdivided into at least three but no more than five dose formulations all having a different combination of vitamins and minerals.

2. The multiple dose vitamin-mineral complex according to claim 1 wherein the dose formulation with vitamin B₁₂ does not contain any of vitamins B₁, C and minerals Cu, Fe, and wherein the dose formulation with mineral Fe does not contain any of minerals Ca, Mg, and Zn, and wherein vitamin D and mineral Ca occur in the same dose formulation.

3. The multiple dose vitamin-mineral complex according to claim 2, wherein mineral Ca and vitamin B₁₂ occur in the same dose formulation.

4. The multiple dose vitamin-mineral complex according to claim 2, wherein mineral Se and vitamin E occur in the same dose formulation.

5. The multiple dose vitamin-mineral complex according to claim 2, wherein vitamins A, C, and E occur in the same dose formulation.

6. The multiple dose vitamin-mineral complex according to claim 2, wherein vitamin K occurs in a dose formulation other than that containing vitamins A and E.

7. The multiple dose vitamin-mineral complex according to claim 3, wherein mineral Se and vitamin E occur in the same dose formulation, and wherein vitamins A, C, and E occur in the same dose formulation, and wherein the dose formulation with vitamin K does not contain vitamins A and E.

8. The multiple dose vitamin-mineral complex according to claim 2, wherein said dose formulations are in the form of liquid drops, capsules, tablets, or lozenges and each dose formulation is a different color.

9. A vitamin-mineral complex to be administered daily comprising:
(a) vitamins A, B₁, B₂, B₆, B₁₂, C, D, E, K, biotin, nicotinamide, pantothenic acid, folic acid; and
(b) minerals Fe, I, Cu, Mo, Mg, Mn, Se, Zn, Ca, Cr;
wherein said vitamin-mineral complex is subdivided into n dose formulations to decrease the number of antagonistic interactions and increase the number of synergistic interactions, where n=3, 4, or 5, and at least three of the dose formulations have a different combination of vitamins and minerals, and wherein vitamin B₁₂ occurs in a dose formulation other than that containing vitamins B., C and minerals Cu, Fe, and wherein mineral Fe occurs in a dose formulation other than that containing minerals Ca, Mg, Zn, and wherein vitamin D and mineral Ca occur in the same dose formulation.

10. The vitamin-mineral complex according to claim 9, wherein mineral Ca and vitamin B12 occur in the same dose formulation.

11. The vitamin-mineral complex according to claim 9, wherein mineral Se and vitamin E occur in the same dose formulation.

12. The vitamin-mineral complex according to claim 9, wherein vitamins A, C, and E occur in the same dose formulation.

13. The vitamin-mineral complex according to claim 9, wherein vitamin K occurs in a dose formulation other than that containing vitamins A and E.

14. The vitamin-mineral complex according to claim 10, wherein mineral Se and vitamin E occur in the same dose formulation, vitamins A, C, and E occur in the same dose formulation, and vitamin K occurs in a dose formulation other than that containing vitamins A and E.

15. A multiple dose vitamin-mineral complex to be administered daily comprising:
(a) vitamins A, B₁, B₁₂, C, D, E, and K; and
(b) minerals Fe, I, Cu, Mg, Se, Zn, and Ca;
wherein said vitamin-mineral complex is subdivided into at least three dose formulations and at least two of the dose formulations have a different combination of vitamins and minerals.

16. The multiple dose vitamin-mineral complex of claim 15, wherein vitamin B₁₂ occurs in a dose formulation other than that containing vitamins B₁, C and minerals Cu, Fe, and wherein mineral Fe occurs in a dose formulation other than that containing minerals Ca, Mg, Zn, and wherein vitamin D and mineral Ca occur in the same dose formulation.

17. The multiple dose vitamin-mineral complex according to claim 16, wherein mineral Ca and vitamin B₁₂ occur in the same dose formulation.

18. The multiple dose vitamin-mineral complex according to claim 16, wherein mineral Se and vitamin E occur in the same dose formulation.

19. The multiple dose vitamin-mineral complex according to claim 15, wherein vitamins A, C, and E occur in the same dose formulation and in a dose formulation other than that containing vitamin K.

20. The multiple dose vitamin-mineral complex according to claim 17, wherein mineral Se and vitamin E occur in the same dose formulation, vitamins A, C, and E occur in the same dose formulation, and vitamin K occurs in a dose formulation other than that containing vitamins A and E.
